(19) United States
(12) Patent Application Publication
(10) Pub. No.: US 2016/0143819 A1
(43) Pub. Date: May 26, 2016

(54) FULLY USEABLE SOAP

(71) Applicant: Neil E. BOHENSKY, Brooklyn, NY (US)

(72) Inventor: Neil E. BOHENSKY, Brooklyn, NY (US)

(21) Appl. No.: 14/952,129

(22) Filed: Nov. 25, 2015

Related U.S. Application Data
(60) Provisional application No. 62/083,948, filed on Nov. 25, 2014.

(51) Int. Cl. A61K 8/02; A61Q 19/10

(52) U.S. Cl. CPC A61K 8/0216; A61Q 19/10; A61K 2800/87

(57) ABSTRACT

An economical soap bar which includes a soap bar body with a hollow interior and an insert occupying the hollow interior, preferably with projecting fingers that penetrate into the soap bar body. The projecting fingers or follicles are designed to penetrate into the body of the soap and provide a massaging sensation to the body by being formed of flexible, hair-like or follicles that are very flexible, or by providing the follicles as combination of flexible fingers, some of which have a rigidity greater than other projecting fingers that are more flexible.

### CROSS-REFERENCE TO RELATED APPLICATION

This application claims benefit of and priority to U.S. Provisional Application Ser. No. 62/083,948 filed Nov. 25, 2014, the contents of which are incorporated herein by reference.

### FIELD OF THE INVENTION

The present invention is directed to soaps and, more particularly, to soaps with a soap insert therein which is effective for maximizing the usage of the soap.

### BACKGROUND OF THE INVENTION

It has been known in the art to utilize a soap insert in order to minimize or eliminate waste in the use of soap owing to soap bars becoming too small to keep from disintegrating or holding in one's hand, even though there was usable soap left.

The prior art has been aware of the use of inserts, per se, for the purpose indicated above. The prior art is exemplified by U.S. Pat. Nos. 1,997,474; 3,773,672; 4,277,358; and 4,279,854, and the contents thereof are incorporated by reference herein.

### SUMMARY OF THE INVENTION

The primary object of the present invention is to provide improvements over known prior art soaps that have been manufactured with inserts therein.

In accordance with one embodiment of the invention, the insert is provided with hard or soft projections preferably made of a synthetic material that serve the dual purpose of allowing the soap bar to adhere to the insert and also to allow massaging the body when the soap is worn down to the insert. It also guides the use of the person's hand to be able to turn and rotate the soap bar to a position on which soap is still left.

In accordance with another embodiment, the insert is an air sack with a very thin and collapsible wall that can be blown up from the outside when the soap is ready to be used, but which has a collapsed position for shipping purposes so as to reduce the product volume during shipping and storage.

Other features and advantages of the present invention will become apparent from the following description of the invention that refers to the accompanying drawings.

### BRIEF DESCRIPTION OF THE DRAWINGS

FIG. 1 is a perspective showing a first embodiment of the invention.

FIGS. 2 and 3 show two product states of a second embodiment of the invention.

FIG. 4 shows a third embodiment of the invention.

### DETAILED DESCRIPTION OF EMBODIMENTS OF THE INVENTION

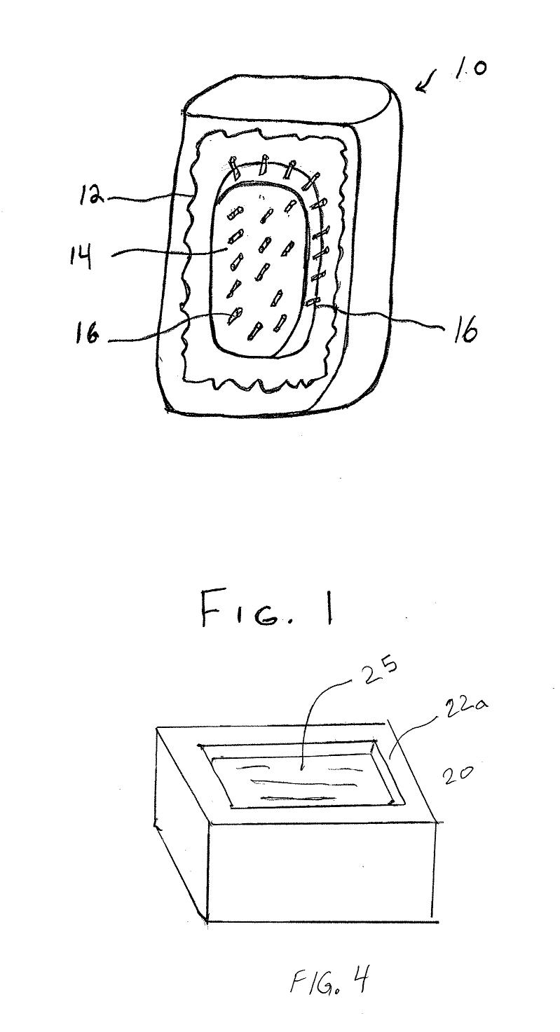

Referring to FIG. 1, in accordance with a first embodiment of the invention, an otherwise conventional bar of soap 10 has a hollow interior that is filled with an insert 14, which preferably also has a hollow interior. The outer shape of the bar of soap preferably is the same as that of insert 14, so that the soap has a somewhat uniform thickness around all sides of the insert embedded during manufacturing. The insert 14 has projecting fingers or follicles 16 which penetrate into the body of the soap 10, as revealed in FIG. 1 through the broken line 12. The fingers 16 can be somewhat rigid or completely flexible, hair-like, or have intermediate flexibility, or combinations of more and less flexible fingers.

As one uses up the bar of soap during hand or body washing, the soap body will slowly wear away until it reaches the insert 14. That will provide an immediate sensation to the body or to the hand via the fingers 16 and one can simply turn the bar of soap to a different orientation, or side or to the rear thereof until the entire body of soap is utilized with no waste whatsoever. The wall of the insert 14 can be made of very thin plastic that itself has microscopic or very small openings with a perfume liquid or gas in the hollow interior, which permeates the body of soap and adds that to the experience of using the bar of soap shown in FIG. 1.

In accordance with a further embodiment of the invention, the insert within the soap bar serves the additional purpose of encouraging young children to want to bathe and soap themselves by forming the insert as a collectible item which can be a figurine, a three-dimensional likeness of ballplayers or a tiny little toy or the like which encourages children to take baths and to soap themselves copiously with great desire of discovering what figurine, toy or trinket is provided within the device.

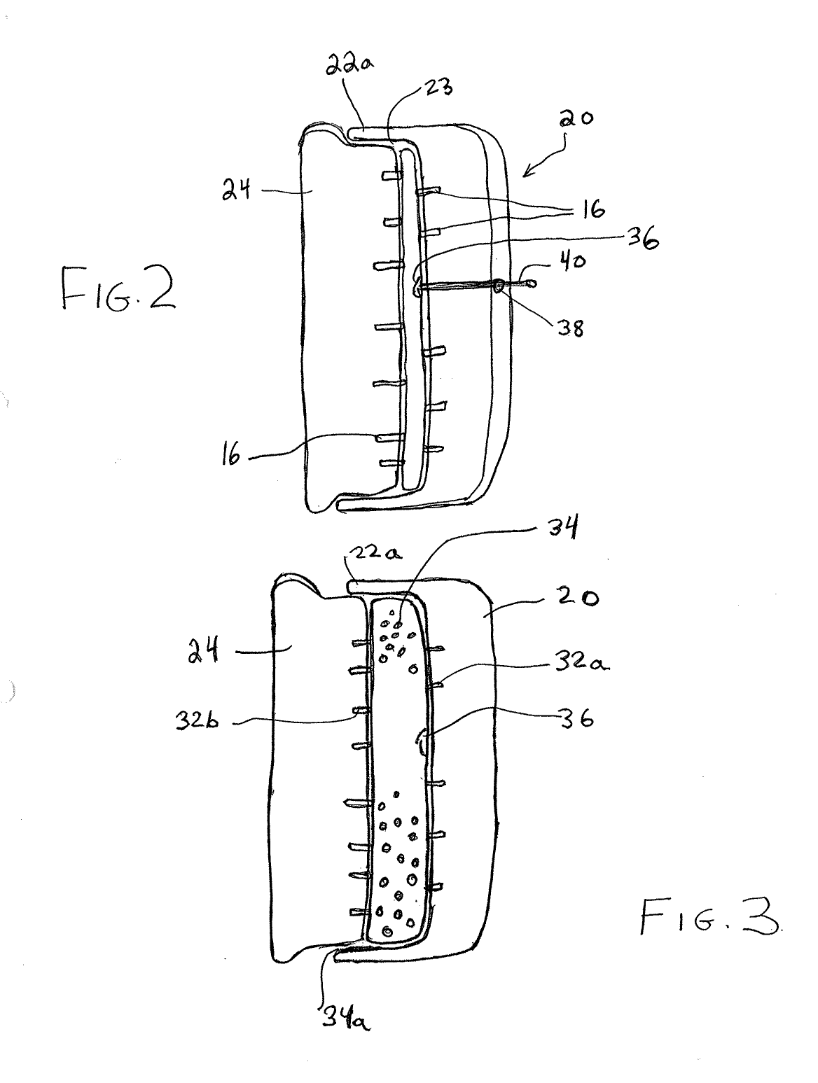

FIGS. 2 and 3 address the concern about increasing the size of the soap package during storage and shipping, which adds an unnecessary cost and inconvenience to the product. To address and alleviate this concern, the soap 20 of FIGS. 2 and 3 comes in two parts including an outer soap 22 with a surrounding peripheral wall or skirt 22a defining a space 23 within which is received a collapsed air sack 40 which has finger projections 32 that are embedded in the soap 22 along one side thereof. The opposing side of the air sack (see FIG. 3) has fingers 32b, which are embedded in an inner soap 24, which fits together as shown in FIG. 2. This sack insert 30 has a one-way air valve 36 and the soap part 22 has a passage 38, which accommodates a windpipe 40, which reaches the valve 36 in the insert, for the purpose described below.

In its collapsed state, the collapsed soap 20 in FIG. 2 is not appreciably larger than an ordinary soap bar. However, when a user wants to use the bar of soap 20, such usage is preceded with blowing air through the air pipe 40, causing the air sack 34 to fill with air and thereby push out the inner soap 24 to the position shown in FIG. 3 with just a small portion of the peripheral skirt 22a still resting on the soap bar 24.

Now the user has a larger sized bar of soap which is easier to hold and use and which provides a better sensation against one's body. The very tight air gap 34a which is inherently present between the skirt 22a of the soap bar 22 and the inner soap bar 24 is inherently sealed after the first time that the expanded soap bar is utilized because of the fusion of the skirt 22a to the body of the soap bar 24, when exposed to water for the first time. The fingers 32a and 32b provide the same function and effect as previously described relative to the first embodiment. The interior of the collapsible sack 30 may also be provided with a scented material to improve and enhance the experience of using the soap bar of the present invention.

An additional advantage of the present invention is that there is no environmentally detrimental large piece of plastic left over, but rather only an air bag, which can be collapsed by being punctured when the use of the soap bar is completed. In addition, the body of the air sack can be made of biodegradable material which is not harmful to the environment, for example, by utilizing a material such as paper or the like.

In accordance a further embodiment of the invention, the soap bar of FIGS. 2 and 3 is provided only with the component 20 whose skirt 22a defines a small receiving section, for example, a bounded space 25 having a depth of about 5 mm to 10 mm, in which a used up, small and thin soap bar which is no longer comfortably held in one hand, is inserted. In other words, the soap is provided with a bounded space that receives a used up and size-diminished bar, whereby when the old and new soap bars are combined, the old and small bar will not slide relative to the new soap bar, allowing thorough usage of the old bar of soap. The perimeter shape of the bounded space can be rectangular, oval, square, etc.

Although the present invention has been described in relation to particular embodiments thereof, many other variations and modifications and other uses will become apparent to those skilled in the art. It is preferred, therefore, that the present invention be limited not by the specific disclosure herein, but only by the appended claim.

What is claimed is:

1. An economical soap bar, comprising:
a soap bar body with a hollow interior, and an insert occupying the hollow interior.

2. The economical soap bar of claim 1, wherein the insert comprising projecting fingers that penetrate into the soap bar body.

3. The economical soap bar of claim 1, wherein the projecting fingers are made of one or more of rigid, flexible and hair-like material.

4. The economical soap bar of claim 2, wherein the insert comprises a thin outer plastic wall and is provided with a fluid that communicates via small openings in the thin plastic into the soap bar body.

5. The economical soap bar of claim 4, wherein the fluid is a perfumed liquid.

6. The economical soap bar of claim 1, wherein the insert comprises one of a figurine, a toy or a trinket.

7. The economical soap bar of claim 1, wherein the soap bar body comprises two body parts, including an outer soap body with a surrounding peripheral wall defining a space;
an inner soap body which fits together with the outer soap body; and
a collapsed and inflatable air sack located between the outer soap body and the inner soap body.

8. The economical soap bar of claim 7, including a one-way air valve that is configured to receive a windpipe that can be utilized to inflate the air sack.

9. An economical soap bar, comprising: a soap bar body having at one side thereof a surrounding and bounding wall that defines a bounded space configured to receive a smaller, leftover soap bar and hold the same in a manner that does not allow the smaller soap bar to slide past the bounding wall.
